(19) United States
(12) Patent Application Publication
(10) Pub. No.: US 2022/0283173 A1
(43) Pub. Date: Sep. 8, 2022

(54) DIFFERENTIAL DETECTION OF VIRAL AND BACTERIAL INFECTIONS

(71) Applicant: ProciseDx Inc., San Diego, CA (US)

(72) Inventors: Larry Mimms, San Diego, CA (US); Michael Hale, San Diego, CA (US)

(73) Assignee: ProciseDx Inc., San Diego, CA (US)

(21) Appl. No.: 17/747,469

(22) Filed: May 18, 2022

Related U.S. Application Data
(63) Continuation of application No. PCT/US2020/063033, filed on Dec. 3, 2020.
(60) Provisional application No. 62/943,530, filed on Dec. 4, 2019.

(51) Int. Cl. G01N 33/68

(52) U.S. Cl. CPC G01N 33/68; G01N 2333/4737; G01N 2333/4715; G01N 2800/26

(57) ABSTRACT

The present disclosure provides assay methods to detect and measure proteins in order to distinguish between a bacterial infection and a viral infection.

### CROSS-REFERENCE TO RELATED APPLICATIONS

This application is a continuation of International Patent Application No. PCT/US2020/063033, filed Dec. 3, 2020, which claims priority to U.S. Provisional Patent Application No. 62/943,530, filed Dec. 4, 2019, the teachings of which are hereby incorporated by reference in their entirety for all purposes.

### BACKGROUND OF THE INVENTION

C-reactive protein is a pentameric protein found in blood plasma, whose circulating concentration rises in response to inflammation. The protein is synthesized by the liver in response to factors released by macrophages and fat cells (adipocytes). C-reactive protein binds to lysophosphatidylcholine expressed on the surface of dead or dying cells (and some types of bacteria) in order to activate the complement system via C1q. The C-reactive protein gene is located on chromosome 1 (1q23.2). Each monomer of its pentameric structure has 224 amino acids, and a molecular mass of 25,106 Da. In serum, it assembles into a stable pentameric structure with a discoid shape. The presence and concentration level of C-reactive protein is typically measured by an enzyme-linked immunosorbent assay (ELISA).

In general, a C-reactive protein solid-phase sandwich ELISA is designed to measure the presence or amount of the analyte bound between an antibody pair. In the sandwich ELISA, a sample is added to an immobilized capture antibody. After a second (detector) antibody is added, a substrate solution is used that reacts with an enzyme-antibody-target complex to produce a measurable signal. The intensity of this signal is proportional to the concentration of target present in the test sample.

The level of C-reactive protein (CRP), which can be measured in the blood, increases with inflammation. C-reactive protein levels can indicate a bacterial infection. For a standard CRP test, a normal reading is less than 10 milligram per liter (mg/L). A test result showing a CRP level greater than 10 mg/L may indicate a bacterial infection, which may require further testing to determine the cause.

Human myxovirus resistance protein A (MxA), a 78 kDa protein, accumulates in the cytoplasm of IFN treated cells and is induced during viral infections. MxA protein may offer certain advantages as a marker for viral infection over other induced proteins such as 2′,5′-oligoadenylate synthetase, because of its lower basal concentration, longer half-life (2.3 days) and fast induction. MxA mRNA is detectable in isolated peripheral white blood cells stimulated with IFN within 1 to 2 h of IFN induction, and MxA protein begins to accumulate shortly thereafter. Studies have shown that MxA protein expression in peripheral blood is a sensitive and specific marker for viral infection. The higher MxA levels in the viral infection group compared with the bacterial infection group can be explained by the fact that the MxA protein is induced exclusively by type I IFN and not by IFN-gamma, IL-1, TNF-alpha, or any of the other cyotokines by bacterial infection. Serum type I IFN levels remain within normal limits, even in patients with bacterial infections.

It remains challenging to differentially diagnosis a bacterial infection over a viral infection. It is difficult to determine the origin of an infection because many ailments such as pneumonia, meningitis, and diarrhea, can be caused by either bacteria or viruses. However, the treatments of a bacterial infection is much different than a viral infection. Thus, there remains a need to differentially diagnose a bacterial infection versus a viral infection. The present disclosure satisfies this and other needs.

### BRIEF SUMMARY OF THE DISCLOSURE

In one embodiment, the present disclosure provides a sandwich assay method for detecting the presence or amount of C-Reactive Protein (CRP) and Myxovirus resistance protein 1 (MxA) in a sample, the method comprising:
contacting the sample with a first anti-CRP antibody having a first binding epitope to CRP, wherein the first anti-CRP antibody is labeled with a first donor fluorophore;
contacting the sample with a second anti-CRP antibody having a second binding epitope to CRP, wherein the second anti-CRP antibody is labeled with a first acceptor fluorophore;
contacting the sample with a first anti-MxA antibody having a first binding epitope to MxA, wherein the first anti-MxA antibody is labeled with a second donor fluorophore;
contacting the sample with a second anti-MxA antibody having a second binding epitope to MxA, wherein the second anti-MxA antibody is labeled with a second acceptor fluorophore;
incubating the sample for a time sufficient to obtain dual labeled CRP and dual labeled MxA; and
exciting the sample having dual labeled CRP and dual labeled MxA using one or more light sources to detect at least one fluorescence emission signal associated with fluorescence resonance energy transfer (FRET), wherein the first and second acceptor fluorophores are different.

In another embodiment, the present disclosure provides an inhibition assay method for detecting the presence or amount of C-Reactive Protein (CRP) and Myxovirus resistance protein 1 (MxA) in a sample, the method comprising:
contacting the sample with a CRP complex comprising an anti-C-reactive protein antibody labeled with a first donor fluorophore and an isolated C-reactive protein labeled with a first acceptor fluorophore, wherein the CRP complex emits a fluorescence emission signal associated with fluorescence resonance energy transfer (FRET) when the first donor fluorophore is excited using a light source;
contacting the sample with a MxA complex comprising an anti-MxA antibody labeled with a second donor fluorophore and an isolated MxA protein labeled with a second acceptor fluorophore, wherein the MxA complex emits a fluorescence emission signal associated with fluorescence resonance energy transfer (FRET) when the donor fluorophore is excited using a light source;
incubating the sample with the CRP complex for a time sufficient for C-reactive protein in the sample to compete for binding to the anti-C-reactive protein antibody labeled with the first donor fluorophore;
incubating the sample with the MxA complex for a time sufficient for MxA protein in the sample to compete for binding to the anti-MxA antibody labeled with the second donor fluorophore; and
exciting the sample using a light source to detect a fluorescence emission signal associated with FRET,
wherein an absence of the fluorescence emission signal or a decrease in the fluorescence emission signal relative to the fluorescence emission signal initially emitted by each of the complexes indicates the presence or amount of C-reactive protein and MxA protein in the sample.

In yet another embodiment, the present disclosure provides a mixed sandwich-inhibition assay method for detecting the presence or amount of C-Reactive Protein (CRP) and Myxovirus resistance protein 1 (MxA) in a sample, the method comprising:
contacting the sample with a first anti-CRP antibody having a first binding epitope to CRP, wherein the first anti-CRP antibody is labeled with a first donor fluorophore;
contacting the sample with a second anti-CRP antibody having a second binding epitope to CRP, wherein the second anti-CRP antibody is labeled with a first acceptor fluorophore;
contacting the sample with a MxA complex comprising an anti-MxA antibody labeled with a second donor fluorophore and an isolated MxA protein labeled with a second acceptor fluorophore, wherein the MxA complex emits a fluorescence emission signal associated with fluorescence resonance energy transfer (FRET) when the second donor fluorophore is excited using a light source;
incubating the sample for a time sufficient to obtain dual labeled CRP;
incubating the sample with the MxA complex for a time sufficient for MxA protein in the sample to compete for binding to the anti-MxA antibody labeled with the second donor fluorophore; and
exciting the sample using a light source to detect a fluorescence emission signal associated with dual labeled CRP and wherein an absence of the fluorescence emission signal or a decrease in the fluorescence emission signal relative to the fluorescence emission signal initially emitted by the MxA complex indicates the presence or amount of MxA protein in the sample.

In still yet another embodiment, the present disclosure provides a mixed inhibition-sandwich assay method for detecting the presence or amount of C-Reactive Protein (CRP) and Myxovirus resistance protein 1 (MxA) in a sample, the method comprising:
contacting the sample with a CRP complex comprising an anti-C-reactive protein antibody labeled with a first donor fluorophore and an isolated C-reactive protein labeled with a first acceptor fluorophore, wherein the CRP complex emits a fluorescence emission signal associated with fluorescence resonance energy transfer (FRET) when the first donor fluorophore is excited using a light source;
contacting the sample with a first anti-MxA antibody having a first binding epitope to MxA, wherein the anti-MxA antibody is labeled with a second donor fluorophore;
contacting the sample with a second anti-MxA antibody having a second binding epitope to MxA, wherein the second anti-MxA antibody is labeled with a second acceptor fluorophore;
incubating the sample with the CRP complex for a time sufficient for C-reactive protein in the sample to compete for binding to the anti-C-reactive protein antibody labeled with the donor fluorophore;
incubating the sample for a time sufficient to obtain dual labeled MxA; and
exciting the sample using a light source to detect a fluorescence emission signal associated with dual labeled MxA and wherein an absence of the fluorescence emission signal or a decrease in the fluorescence emission signal relative to the fluorescence emission signal initially emitted by the CRP complex indicates the presence or amount of CRP in the sample.

These and other aspects, objects, and embodiments will become more apparent when read with the detailed description and figures that follow.

### BRIEF DESCRIPTION OF THE DRAWINGS

FIGS. 1A-1B illustrate a CRP-MxA multiplex sandwich assay of the present disclosure.

FIGS. 2A-2B illustrates a CRP-MxA multiplex competition assay of the present disclosure.

FIG. 3A illustrates a standard curve generated using methods of the present disclosure.

FIG. 3B illustrates the TR-FRET C-reactive protein assay reaches equilibrium after 1 minute. Four concentrations (0, 5, 10, and 50 mg/mL) of C-reactive protein in the sample were tested. Reagents were mixed with the sample and FRET signals were read at different time points as shown.

FIG. 3C illustrates a standard curve generated using methods of the present disclosure.

FIG. 4 illustrates one embodiment of a donor fluorophore of the present disclosure.

FIG. 5 illustrates one embodiment of an acceptor fluorophore of the present disclosure.

FIG. 6 illustrates donor and acceptor wavelengths in one embodiment of the present disclosure.

### DETAILED DESCRIPTION OF THE DISCLOSURE

I. Definitions

The terms “a,” “an,” or “the” as used herein not only includes aspects with one member, but also includes aspects with more than one member.

The term “about” as used herein to modify a numerical value indicates a defined range around that value. If “X” were the value, “about X” would indicate a value from 0.9X to 1.1X, and more preferably, a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

When the modifier “about” is applied to describe the beginning of a numerical range, it applies to both ends of the range. Thus, “from about 500 to 850 nm” is equivalent to “from about 500 nm to about 850 nm.” When “about” is applied to describe the first value of a set of values, it applies to all values in that set. Thus, “about 580, 700, or 850 nm” is equivalent to “about 580 nm, about 700 nm, or about 850 nm.”

“Activated acyl” as used herein includes a —C(O)—LG group. “Leaving group” or “LG” is a group that is susceptible to displacement by a nucleophilic acyl substitution (i.e., a nucleophilic addition to the carbonyl of —C(O)—LG, followed by elimination of the leaving group). Representative leaving groups include halo, cyano, azido, carboxylic acid derivatives such as t-butylcarboxy, and carbonate derivatives such as i-BuOC(O)O—. An activated acyl group may also be an activated ester as defined herein or a carboxylic acid activated by a carbodiimide to form an anhydride (preferentially cyclic) or mixed anhydride —OC(O)R^{a} or —OC(NR^{a})NHR^{b} (preferably cyclic), wherein R^{a} and R^{b} are members independently selected from the group consisting of C₁-C₆ alkyl, C₁-C₆ perfluoroalkyl, C₁-C₆ alkoxy, cyclohexyl, 3-dimethylaminopropyl, or N-morpholinoethyl. Preferred activated acyl groups include activated esters.

“Activated ester” as used herein includes a derivative of a carboxyl group that is more susceptible to displacement by nucleophilic addition and elimination than an ethyl ester group (e.g., an NHS ester, a sulfo-NHS ester, a PAM ester, or a halophenyl ester). Representative carbonyl substituents of activated esters include succinimidyloxy (—OC₄H₄NO₂), sulfosuccinimidyloxy (—OC₄H₃NO₂SO₃H), -1-oxybenzotriazolyl (—OC₆H₄N₃); 4-sulfo-2,3,5,6-tetrafluorophenyl; or an aryloxy group that is optionally substituted one or more times by electron-withdrawing substituents such as nitro, fluoro, chloro, cyano, trifluoromethyl, or combinations thereof (e.g., pentafluorophenyloxy, or 2,3,5,6-tetrafluorophenyloxy). Preferred activated esters include succinimidyloxy, sulfosuccinimidyloxy, and 2,3,5,6-tetrafluorophenyloxy esters.

“FRET partners” refers to a pair of fluorophores consisting of a donor fluorescent compound such as cryptate and an acceptor compound such as Alexa 647, when they are in proximity to one another and when they are excited at the excitation wavelength of the donor fluorescent compound, these compounds emit a FRET signal. It is known that, in order for two fluorescent compounds to be FRET partners, the emission spectrum of the donor fluorescent compound must partially overlap the excitation spectrum of the acceptor compound. The preferred FRET-partner pairs are those for which the value RO (Förster distance, distance at which energy transfer is 50% efficient) is greater than or equal to 30 Å.

“FRET signal” refers to any measurable signal representative of FRET between a donor fluorescent compound and an acceptor compound. A FRET signal can therefore be a variation in the intensity or in the lifetime of luminescence of the donor fluorescent compound or of the acceptor compound when the latter is fluorescent.

“C-reactive protein” or CRP refers to a pentameric protein found in the blood plasma, whose circulating concentrations rise in response to inflammation. The protein is synthesized by the liver in response to factors released by macrophages and fat cells (adipocytes). The C-reactive protein gene is located on chromosome 1 (1q23.2). Each monomer of its pentameric structure has 224 amino acids, and a molecular mass of 25,106 Da. In serum, it assembles into stable pentameric structure with a discoid shape. Human C-reactive protein, UniProt ID No. P02741, is SEQ ID NO: 1.

“Human myxovirus resistance protein A” (MxA) the product of the MX1 gene, is a 76-kDa protein consisting of 662 amino acid residues and belonging to the dynamic superfamily of large GTPase. MxA protein plays an important role in antiviral activity in cells against a wide variety of viruses, including influenza, parainfluenza, measles, coxsackie, hepatitis B virus, and Thogoto virus. The viruses are inhibited by MxA protein at an early stage in their life cycle, soon after host cell entry and before genome amplification. The mouse MxA (MX1 GTPase) accumulates in the cell nucleus where it associates with nuclear bodies and inhibits influenza and Thogoto viruses known to replicate in the nucleus. The human MxA protein accumulates in the cytoplasm and endoplasmic reticulum as well.

Human MxA is 662 amino acids (aa) in length (UniProt ID NO: P20591-1, SEQ ID NO:2). It contains one GTPase domain (aa 69-340) and a GED (GTPase Effector Domain) over aa 574-662. There are two utilized phosphorylation sites at Tyr129 and Tyr451. MxA forms homo-dimers, -tetramers and -oligomers, with multimerization suggested to be important for activity. Although IFNs are typically considered to induce Mx gene expression, HSV-1 itself also activates gene transcription. In this case, however, a truncated 54-57 kDa transcript is generated that contains an 84 aa substitution for aa 425-662. With respect to aa 412-630 (over aa 412-630), human MxA shares 49% aa sequence identity with mouse Mx1.

True positive “TP” means positive test result that accurately reflects the tested-for activity. For example in the context of the present disclosure a TP, is for example but not limited to, truly classifying a bacterial infection as such.

True negative “TN” means a negative test result that accurately reflects the tested-for activity. For example in the context of the present disclosure a TN, is for example but not limited to, truly classifying a viral infection as such.

False negative “FN” means a result that appears negative but fails to reveal a situation. For example in the context of the present disclosure a FN, is for example but not limited to, falsely classifying a bacterial infection as a viral infection.

False positive “FP” means a test result that is erroneously classified in a positive category. For example in the context of the present disclosure, a FP, is for example but not limited to, falsely classifying a viral infection as a bacterial infection.

Sensitivity is calculated by TP/(TP+FN) or the true positive fraction of disease subjects.

Specificity is calculated by TN/(TN+FP) or the true negative fraction of non-disease or normal subjects.

Total accuracy is calculated by (TN+TP)/(TN+FP+TP+FN).

Positive predictive value or “PPV” is calculated by TP/(TP+FP) or the true positive fraction of all positive test results.

Negative predictive value or “NPV” is calculated by TN/(TN+FN) or the true negative fraction of all negative test results.

II. Embodiments

The present disclosure provides a method for measuring CRP and MxA concentration levels to differentially detect and or diagnose a bacterial infection versus a viral infection. The assay method can be performed in multiplex fashion using the same sample to simultaneously detect and measure two or more analytes, or in parallel or sequential individual assays. In one embodiment, the assay is a multiplex assay measuring both CRP and MxA analytes being bound by their respective antibody pairs.

As such, in one embodiment, the present disclosure provides a sandwich assay method for detecting the presence or amount of C-Reactive Protein (CRP) and Myxovirus resistance protein 1 (MxA) in a sample, the method comprising:
contacting the sample with a first anti-CRP antibody having a first binding epitope to CRP, wherein the first anti-CRP antibody is labeled with a first donor fluorophore;
contacting the sample with a second anti-CRP antibody having a second binding epitope to CRP, wherein the second anti-CRP antibody is labeled with a first acceptor fluorophore;
contacting the sample with a first anti-MxA antibody having a first binding epitope to MxA, wherein the first anti-MxA antibody is labeled with a second donor fluorophore;
contacting the sample with a second anti-MxA antibody having a second binding epitope to MxA, wherein the second anti-MxA is labeled with a second acceptor fluorophore;
incubating the sample for a time sufficient to obtain dual labeled CRP and dual labeled MxA; and
exciting the sample having dual labeled CRP and dual labeled MxA using one or more light sources to detect at least one fluorescence emission signal associated with fluorescence resonance energy transfer (FRET), wherein the first and second acceptor fluorophores are different.

Advantageously, in one aspect of the present disclosure, the same donor (such as a cryptate dye) can be used for an anti-CRP antibody and an anti-MxA antibody. In other words, in some embodiments, the first and second donor fluorophores are the same and the sample is excited using one light source. In other embodiments of the disclosure, the first and second donor fluorophores are different and the sample is excited using two different light sources.

The assay format may also be performed in competition format. In the above multiplex format, each analyte is bound a pair of antibodies in a sandwich format. Alternatively, the assay can be performed in competition format wherein endogenous protein competes with labeled protein. In this manner, a fluorescence emission signal associated with labeled protein is inversely proportional to the concentration level of endogenous protein.

As such, in one embodiment, the present disclosure provides an inhibition assay method for detecting the presence or amount of C-Reactive Protein (CRP) and Myxovirus resistance protein 1 (MxA) in a sample, the method comprising:
contacting the sample with a CRP complex comprising an anti-C-reactive protein antibody labeled with a first donor fluorophore and an isolated C-reactive protein labeled with a first acceptor fluorophore, wherein the CRP complex emits a fluorescence emission signal associated with fluorescence resonance energy transfer (FRET) when the first donor fluorophore is excited using a light source;
contacting the sample with a MxA complex comprising an anti-MxA antibody labeled with a second donor fluorophore and an isolated MxA protein labeled with a second acceptor fluorophore, wherein the MxA complex emits a fluorescence emission signal associated with fluorescence resonance energy transfer (FRET) when the donor fluorophore is excited using a light source;
incubating the sample with the CRP complex for a time sufficient for C-reactive protein in the sample to compete for binding to the anti-C-reactive protein antibody labeled with the first donor fluorophore;
incubating the sample with the MxA complex for a time sufficient for MxA protein in the sample to compete for binding to the anti-MxA antibody labeled with the second donor fluorophore; and
exciting the sample using a light source to detect a fluorescence emission signal associated with FRET,
wherein an absence of the fluorescence emission signal or a decrease in the fluorescence emission signal relative to the fluorescence emission signal initially emitted by each of the complexes indicates the presence or amount of C-reactive protein and MxA protein in the sample.

The assay format can also be performed in a mixed competition-sandwich or mixed sandwich-competition format. In the mixed format, one analyte is bound to a pair of antibodies in a sandwich format. The other analyte is measured in a competition format wherein endogenous protein competes with labeled protein. In this manner (competition format), a fluorescence emission signal associated with labeled protein is inversely proportional to endogenous protein level or concentration.

As such, the present disclosure provides a mixed sandwich-inhibition assay method for detecting the presence or amount of C-Reactive Protein (CRP) and Myxovirus resistance protein 1 (MxA) in a sample, the method comprising:
contacting the sample with a first anti-CRP antibody having a first binding epitope to CRP, wherein the first anti-CRP antibody is labeled with a first donor fluorophore;
contacting the sample with a second anti-CRP antibody having a second binding epitope to CRP, wherein the second anti-CRP antibody is labeled with a first acceptor fluorophore;
contacting the sample with a MxA complex comprising an anti-MxA antibody labeled with a second donor fluorophore and an isolated MxA protein labeled with a second acceptor fluorophore, wherein the MxA complex emits a fluorescence emission signal associated with fluorescence resonance energy transfer (FRET) when the second donor fluorophore is excited using a light source;
incubating the sample for a time sufficient to obtain dual labeled CRP;

incubating the sample with the MxA complex for a time sufficient for MxA protein in the sample to compete for binding to the anti-MxA antibody labeled with the second donor fluorophore; and
exciting the sample using a light source to detect a fluorescence emission signal associated with dual labeled CRP and wherein an absence of the fluorescence emission signal or a decrease in the fluorescence emission signal relative to the fluorescence emission signal initially emitted by the MxA complex indicates the presence or amount of MxA protein in the sample.

In yet another embodiment, the present disclosure provides a mixed inhibition-sandwich assay method for detecting the presence or amount of C-Reactive Protein (CRP) and Myxovirus resistance protein 1 (MxA) in a sample, the method comprising:
contacting the sample with a CRP complex comprising an anti-C-reactive protein antibody labeled with a first donor fluorophore and an isolated C-reactive protein labeled with a first acceptor fluorophore, wherein the CRP complex emits a fluorescence emission signal associated with fluorescence resonance energy transfer (FRET) when the first donor fluorophore is excited using a light source;
contacting the sample with a first anti-MxA antibody having a first binding epitope to MxA, wherein the anti-MxA antibody is labeled with a second donor fluorophore;
contacting the sample with a second anti-MxA antibody having a second binding epitope to MxA, wherein the second anti-MxA antibody is labeled with a second acceptor fluorophore;
incubating the sample with the CRP complex for a time sufficient for C-reactive protein in the sample to compete for binding to the anti-C-reactive protein antibody labeled with the donor fluorophore;
incubating the sample for a time sufficient to obtain dual labeled MxA; and
exciting the sample using a light source to detect a fluorescence emission signal associated with dual labeled MxA and wherein an absence of the fluorescence emission signal or a decrease in the fluorescence emission signal relative to the fluorescence emission signal initially emitted by the CRP complex indicates the presence or amount of CRP in the sample.

As is apparent to those of skill in the art, the methods herein can be performed in a multiplex fashion. Alternatively, each of the analytes can be detected and measured individually and in a serial or a simultaneous separate assay fashion.

Förster resonance energy transfer or fluorescence resonance energy transfer (FRET) is a process in which a donor molecule in an excited state transfers its excitation energy through dipole-dipole coupling to an acceptor fluorophore, when the two molecules are brought into close proximity, typically less than 10 nm such as, <9 nm, <8 nm, <7 nm, <6 nm, <5 nm, <4 nm, <3 nm, <2 nm, or less than <1 nm. Upon excitation at a characteristic wavelength, the energy absorbed by the donor is transferred to the acceptor, which in turn emits the energy. The level of light emitted from the acceptor fluorophore is proportional to the degree of donor acceptor complex formation.

Biological materials are typically prone to autofluorescence, which can be minimized by utilizing time-resolved fluorometry (TRF). TRF takes advantage of unique rare earth elements such as lanthanides, (e.g., europium and terbium), which have exceptionally long fluorescence emission half-lives. Time-resolved FRET (TR-FRET) unites the properties of TRF and FRET, which is especially advantageous when analyzing biological samples. If one anti-CRP antibody is labeled with a donor fluorophore and a second anti-CRP antibody is labeled with an acceptor fluorophore, and a first anti-MxA antibody is labeled with a donor fluorophore (or an acceptor fluorophore) and a second anti-MxA antibody is labeled with an acceptor fluorophore (or a donor fluorophore), in which the two acceptor fluorophores are different, TR-FRET can occur in the presence of CRP and MxA in the sample.

The use of the FRET phenomenon for studying biological processes implies that each member of the pair of FRET partners will be conjugated to compounds that will interact with one another, and thus bring the FRET partners into close proximity with one another. Upon exposure to light, the FRET partners will generate a FRET signal. In certain embodiments, an energy donor and an energy acceptor are each conjugated to a different anti-CRP antibody. An energy donor or an energy acceptor is conjugated to a first anti-MxA antibody. Further, an energy donor or an energy acceptor is conjugated to a second anti-MxA antibody. For example, two anti-CRP antibodies that bind to two different epitopes on CRP, and two anti-MxA antibodies that bind to two different epitopes on MxA can be used. The energy transfer between the two FRET partners depends upon each binding to the analyte. Förster or fluorescence resonance energy transfer (FRET), is a physical phenomenon in which a donor fluorophore in its excited state non-radiatively transfers its excitation energy to a neighboring acceptor fluorophore, thereby causing the acceptor to emit its characteristic fluorescence.

In one aspect, two anti-CRP antibodies, one labeled with a donor fluorophore and one labeled with an acceptor fluorophore, are used. The two anti-CRP antibodies bind to two different epitopes on CRP. Similarly, two anti-MxA antibodies are used. One anti-MxA antibody is labeled with a donor fluorophore and one anti-MxA antibody is labeled with an acceptor fluorophore. The two anti-MxA antibodies bind to two different epitopes on MxA. In one aspect in the present disclosure, the same donor (such as a cryptate dye) can be used for an anti-CRP antibody and an anti-MxA antibody. In one aspect in the present disclosure, different donors are used for an anti-CRP antibody and an anti-MxA antibody.

In one aspect, two anti-CRP antibodies binding to two different epitopes on CRP bring the first donor fluorophore and the first acceptor fluorophore in proximity to each other. Likewise, two anti-MxA antibodies binding to two different epitopes on MxA bring the second donor fluorophore and the second acceptor fluorophore in proximity to each other. The donor fluorophore in its excited state can transfer its excitation energy to the acceptor fluorophore to cause the acceptor fluorophore to emit its characteristic fluorescence. In some embodiments, the two acceptor fluorophores are different and emit fluorescence at different wavelengths. Thus, the appearance of the first fluorescence emission signal is proportional to the presence or level of CRP in the sample and the appearance of the second fluorescence emission signal is proportional to the presence or level of MxA in the sample.

In some embodiments of the disclosure, the methods described herein further comprise detecting the presence or amount of an additional biomarker. The measurement of the additional biomarker can be performed in multiplex fashion, wherein the additional biomarker is measure in the same sample simultaneously. Alternatively, the third biomarker is measured before or after CRP and MxA levels are measured. To detect the additional biomarker, the methods comprise:
contacting the sample with an additional antibody having a first binding epitope to the additional biomarker, wherein the additional antibody is labeled with a third donor fluorophore;
contacting the sample with a further antibody having a second binding epitope to the additional biomarker, wherein the further antibody is labeled with a third acceptor fluorophore;
incubating the sample for a time sufficient to obtain dual labeled additional biomarker; and exciting the sample having dual labeled additional biomarker using a light source to detect two fluorescence emission signals associated with fluorescence resonance energy transfer (FRET), wherein the first, second, and third acceptor fluorophores are different.

For example, in some embodiments, the first acceptor fluorophore is Alexa Fluor 488, the second acceptor fluorophore is Alexa Fluor 546, and the third acceptor fluorophore is Alexa Fluor 647. Those of skill in the art will know of other acceptor fluorophores suitable for use in the present disclosure.

In one aspect, the additional biomarker is procalcitonin. Procalcitonin is a substance produced by many types of cells in the body, often in response to bacterial infections but also in response to tissue injury. The level of procalcitonin (PCT) in the blood can increase significantly in systemic bacterial infections and sepsis. The reference value of PCT in adults and children is about 0.15 ng/mL.

In certain aspects, the FRET assay is a time-resolved FRET assay. The fluorescence emission signal or measured FRET signal is directly correlated with the biological phenomenon studied. In fact, the level of energy transfer between the donor fluorescent compound and the acceptor fluorescent compound is proportional to the reciprocal of the distance between these compounds to the 6^{th} power. For the donor/acceptor pairs commonly used by those skilled in the art, the distance Ro (corresponding to a transfer efficiency of 50%) is in the order of 1, 5, 10, 20 or 30 nanometers.

In certain aspects, the sample is a biological sample. Suitable biological samples include, but are not limited to, whole blood, urine, a fecal specimen, plasma or serum. In a preferred aspect, the biological sample is whole blood.

In certain aspects, the FRET energy donor compound (the first donor or the second donor or both) is a cryptate, such as a lanthanide cryptate.

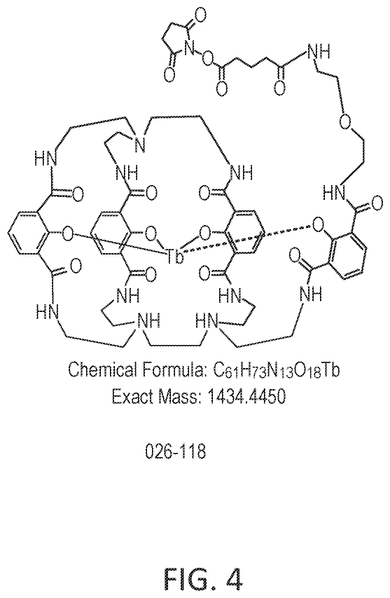

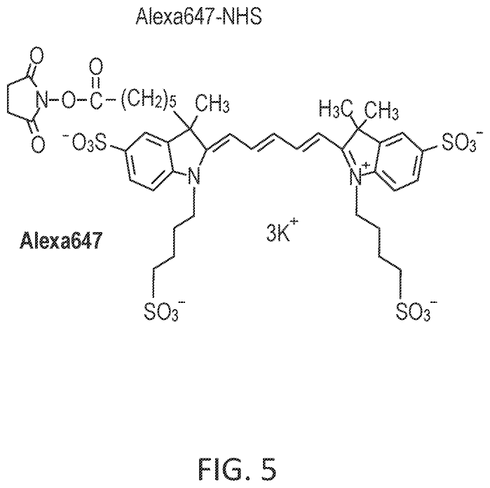

In certain aspects, the cryptate has an absorption wavelength between about 300 nm to about 400 nm such as about 325 nm to about 375 nm. In certain aspects, as shown in FIG. 4, cryptate dyes (Lumi4-Tb in FIG. 4) have four fluorescence emission peaks at about 490 nm, about 548 nm, about 587 nm, and 621 nm. Thus, as a donor, the cryptate is compatible with fluorescein-like (green zone) and Cy5 or DY-647-like (red zone) acceptor (e.g., green acceptor, NIR acceptor, or orange acceptor in FIG. 5) to perform TR-FRET experiments.

In certain aspects, the introduction of a time delay between a flash excitation and the measurement of the fluorescence at the acceptor emission wavelength allows to discriminate long lived from short-lived fluorescence and to increase signal-to-noise ratio.

1. Cryptates as FRET Donors

In certain aspects, the terbium cryptate molecule “Lumi4-Tb” from Lumiphore, marketed by Cisbio bioassays is used as the cryptate donor. The terbium cryptate “Lumi4-Tb” having the formula below, which can be coupled to an antibody by a reactive group, in this case, for example, an NHS ester:

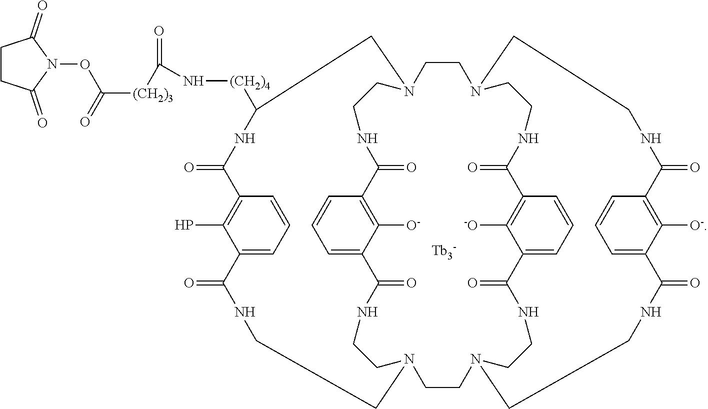

An activated ester (an NHS ester) can react with a primary amine on an antibody to make a stable amide bond. A maleimide on the cryptate and a thiol on the antibody can react together and make a thioether. Alkyl halides react with amines and thiols to make alkylamines and thioethers, respectively. Any derivative providing a reactive moiety that can be conjugated to an antibody can be utilized herein. For example, in some embodiments, when an anti-C-reactive protein antibody is used, the maleimide on the cryptate can react with a thiol on the antibody.

In certain other aspects, cryptates disclosed in WO2015157057, titled “Macrocycles” are suitable for use in the present disclosure. This publication contains cryptate molecules useful for labeling biomolecules. As disclosed therein, certain of the cryptates have the structure:

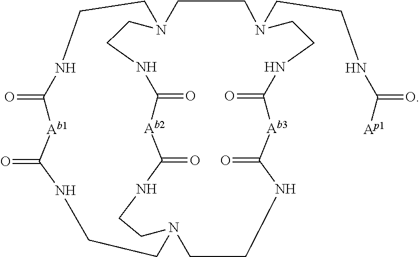

In certain other aspects, a terbium cryptate useful in the present disclosure is shown below:

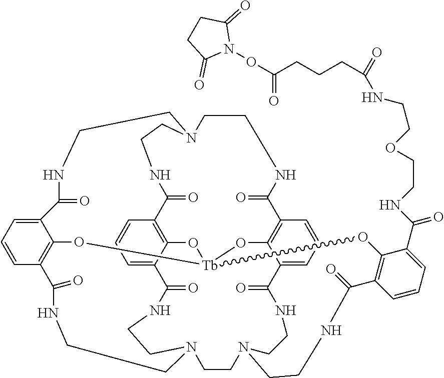

In certain aspects, the cryptates that are useful in the present invention are disclosed in WO 2018/130988, published Jul. 19, 2018. As disclosed therein, the compounds of Formula I are useful as FRET donors in the present disclosure:

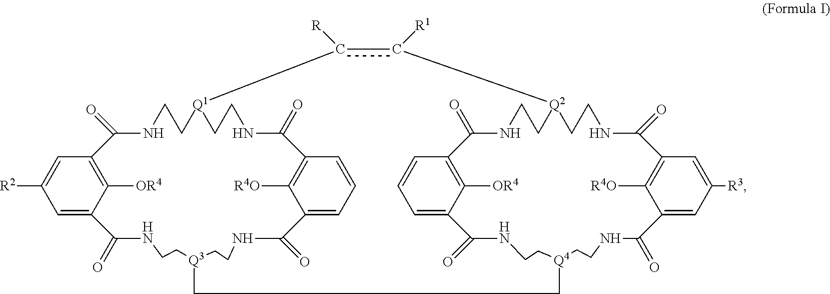

wherein when the dotted line is present, R and le are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and le join to form an optionally substituted cyclopropyl group wherein the dotted bond is absent;

R² and R³ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, or an activated group that can be linked to a biomolecule, wherein the activated group is a member selected from the group consisting of a halogen, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group or L;

R⁴ are each independently a hydrogen, C₁-C₆ alkyl, or alternatively, 3 of the R⁴ groups are absent and the resulting oxides are chelated to a lanthanide cation; and Q¹-Q⁴ are each independently a member selected from the group of carbon or nitrogen.

2. FRET Acceptors

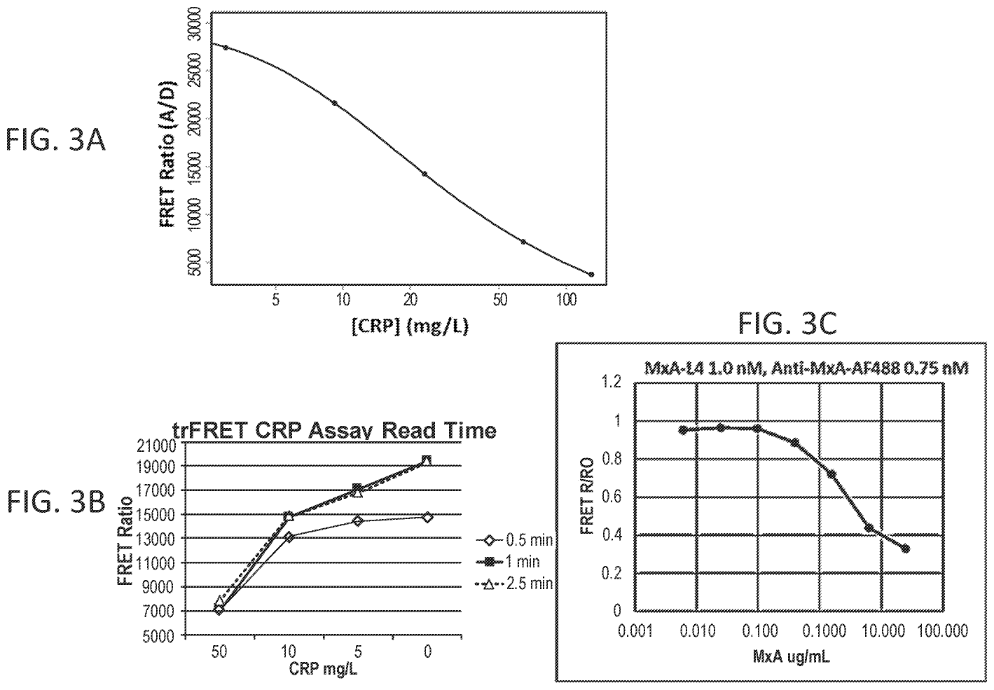

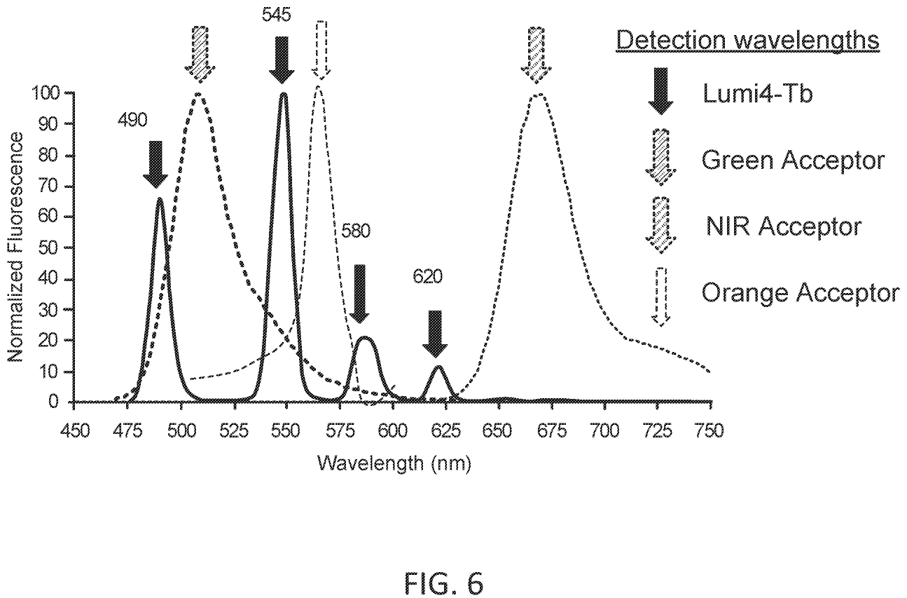

In order to detect a FRET signal, a FRET acceptor is required. The FRET acceptor has an excitation wavelength that overlaps with an emission wavelength of the FRET donor. In the present disclosure, a FRET signal of the acceptor is detected when an anti-C-reactive protein antibody labeled with a donor fluorophore (or an acceptor fluorophore) binds to an isolated C-reactive protein labeled with an acceptor fluorophore (or a donor fluorophore). A known amount of calibrators, i.e., standard curve (FIG. 3A), can be used to interpolate the concentration levels of C-reactive protein in a sample. As described above, the cryptate donor (FIG. 4) can be used to label the anti-C-reactive protein antibody or anti-MxA antibody. Lumi4 has 4 spectrally distinct peaks, at about 490 nm, about 545 nm, about 580 nm, and about 620 nm, which can be used for energy transfer (FIG. 6). Subsequently, a first acceptor can be used to label an anti-C-reactive protein antibody. The acceptor molecules that can be used include, but are not limited to, fluorescein-like (green zone), Cy5, DY-647, Alexa Fluor 488, Alexa Fluor 546, Alexa Fluor 647 (FIG. 5), allophycocyanin (APC), and phycoerythrin (PE). Donor and acceptor fluorophores can be conjugated using a primary amine on an antibody.

Other acceptors include, but are not limited to, cyanin derivatives, D2, CYS, fluorescein, coumarin, rhodamine, carbopyronine, oxazine and its analogs, Alexa Fluor fluorophores, Crystal violet, perylene bisimide fluorophores, squaraine fluorophores, boron dipyrromethene derivatives, NBD (nitrobenzoxadiazole) and its derivatives, and DABCYL (4-((4-(dimethylamino)phenyl)azo)benzoic acid).

In one aspect, fluorescence can be characterized by for example, one or more of the following, wavelength, intensity, lifetime, and polarization.

3. Antibodies

In one aspect, an anti-C-reactive protein antibody (e.g., Catalog # ab31156 (Abcam), and shown to be specific for C-reactive protein) can be used to conjugate to a donor fluorophore (e.g., cryptate) or an acceptor fluorophore. Other commercial anti-C-reactive protein antibodies are available in the art, such as Catalog # ab32412 (Biocompare) and Catalog #MAB17071 (R&D Systems).

In one aspect, an anti-MxA antibody (e.g., Catalog # Anti-MX1 antibody [EPR19967] (ab207414) (Abcam), and shown to be specific for MxA protein) can be used to conjugate to a donor fluorophore (e.g., cryptate) or an acceptor fluorophore. In another aspect, Anti-MX1 antibody [EPR19967] (Alexa Fluor® 488) (ab237298) can be used. Alterntively, Anti-MX1 antibody [EPR19967] (Alexa Fluor® 647) (ab237299) (Abcam) can be used.

The methods herein for detecting the presence or levels of C-reactive and MxA proteins can use a variety of samples, which include a tissue sample, blood, biopsy, serum, plasma, saliva, urine, or stool sample.

4. Production of Antibodies

The generation and selection of antibodies not already commercially available can be accomplished several ways. For example, one way is to express and/or purify a polypeptide of interest (i.e., antigen) using protein expression and purification methods known in the art, while another way is to synthesize the polypeptide of interest using solid phase peptide synthesis methods known in the art. See, e.g., Guide to Protein Purification, Murray P. Deutcher, ed., Meth. Enzymol., Vol. 182 (1990); Solid Phase Peptide Synthesis, Greg B. Fields, ed., Meth. Enzymol., Vol. 289 (1997); Kiso et al., Chem. Pharm. Bull., 38:1192-99 (1990); Mostafavi et al., Biomed. Pept. Proteins Nucleic Acids, 1:255-60, (1995); and Fujiwara et al., Chem. Pharm. Bull., 44:1326-31 (1996). The purified or synthesized polypeptide can then be injected, for example, into mice or rabbits, to generate polyclonal or monoclonal antibodies. One skilled in the art will recognize that many procedures are available for the production of antibodies, for example, as described in Antibodies, A Laboratory Manual, Harlow and Lane, Eds., Cold Spring Harbor Laboratory, Cold Spring Harbor, N.Y. (1988). One skilled in the art will also appreciate that binding fragments or Fab fragments which mimic antibodies can also be prepared from genetic information by various procedures (see, e.g., Antibody Engineering: A Practical Approach, Borrebaeck, Ed., Oxford University Press, Oxford (1995); and Huse et al., J. Immunol., 149:3914-3920 (1992)).

In addition, numerous publications have reported the use of phage display technology to produce and screen libraries of polypeptides for binding to a selected target antigen (see, e.g, Cwirla et al., Proc. Natl. Acad. Sci. USA, 87:6378-6382 (1990); Devlin et al., Science, 249:404-406 (1990); Scott et al., Science, 249:386-388 (1990); and Ladner et al., U.S. Pat. No. 5,571,698). A basic concept of phage display methods is the establishment of a physical association between a polypeptide encoded by the phage DNA and a target antigen. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target antigen bind to the target antigen and these phage are enriched by affinity screening to the target antigen. The identity of polypeptides displayed from these phage can be determined from their respective genomes. Using these methods, a polypeptide identified as having a binding affinity for a desired target antigen can then be synthesized in bulk by conventional means (see, e.g., U.S. Pat. No. 6,057,098).

The antibodies that are generated by these methods can then be selected by first screening for affinity and specificity with the purified polypeptide antigen of interest and, if required, comparing the results to the affinity and specificity of the antibodies with other polypeptide antigens that are desired to be excluded from binding. The screening procedure can involve immobilization of the purified polypeptide antigens in separate wells of microtiter plates. The solution containing a potential antibody or group of antibodies is then placed into the respective microtiter wells and incubated for about 30 minutes to 2 hours. The microtiter wells are then washed and a labeled secondary antibody (e.g., an anti-mouse antibody conjugated to alkaline phosphatase if the raised antibodies are mouse antibodies) is added to the wells and incubated for about 30 minutes and then washed. Substrate is added to the wells and a color reaction will appear where antibody to the immobilized polypeptide antigen is present.

The antibodies so identified can then be further analyzed for affinity and specificity.

In the development of immunoassays for a target protein (C-reactive protein), the purified target protein acts as a standard with which to judge the sensitivity and specificity of the immunoassay using the antibodies that have been selected. Because the binding affinity of various antibodies may differ, e.g., certain antibody combinations may interfere with one another sterically, assay performance of an antibody may be a more important measure than absolute affinity and specificity of that antibody.

Those skilled in the art will recognize that many approaches can be taken in producing antibodies or binding fragments and screening and selecting for affinity and specificity for the various polypeptides of interest, but these approaches do not change the scope of the present invention.

4. Polyclonal Antibodies

Polyclonal antibodies are preferably raised in animals by multiple subcutaneous (sc) or intraperitoneal (ip) injections of a polypeptide of interest and an adjuvant. It may be useful to conjugate the polypeptide of interest to a protein carrier that is immunogenic in the species to be immunized, such as, e.g., keyhole limpet hemocyanin, bovine thyroglobulin, or soybean trypsin inhibitor using a bifunctional or derivatizing agent. Non-limiting examples of bifunctional or derivatizing agents include maleimidobenzoyl sulfosuccinimide ester (conjugation through cysteine residues), N-hydroxysuccinimide (conjugation through lysine residues), glutaraldehyde, succinic anhydride, SOCl₂, and RiN═C═NR, wherein R and R₁ are different alkyl groups.

Animals are immunized against the polypeptide of interest or an immunogenic conjugate or derivative thereof by combining, e.g., 100 μg (for rabbits) or 5 μg (for mice) of the antigen or conjugate with 3 volumes of Freund's complete adjuvant and injecting the solution intradermally at multiple sites. One month later, the animals are boosted with about ⅕ to 1/10 the original amount of polypeptide or conjugate in Freund's incomplete adjuvant by subcutaneous injection at multiple sites. Seven to fourteen days later, the animals are bled and the serum is assayed for antibody titer. Animals are typically boosted until the titer plateaus. Preferably, the animal is boosted with the conjugate of the same polypeptide, but conjugation to a different immunogenic protein and/or through a different cross-linking reagent may be used. Conjugates can also be made in recombinant cell culture as fusion proteins. In certain instances, aggregating agents such as alum can be used to enhance the immune response.

5. Monoclonal Antibodies

Monoclonal antibodies are generally obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, monoclonal antibodies can be made using the hybridoma method described by Kohler et al., Nature, 256:495 (1975) or by any recombinant DNA method known in the art (see, e.g., U.S. Pat. No. 4,816,567).

In the hybridoma method, a mouse or other appropriate host animal (e.g., hamster) is immunized as described above to elicit lymphocytes that produce or are capable of producing antibodies which specifically bind to the polypeptide of interest used for immunization. Alternatively, lymphocytes are immunized in vitro. The immunized lymphocytes are then fused with myeloma cells using a suitable fusing agent, such as polyethylene glycol, to form hybridoma cells (see, e.g., Goding, Monoclonal Antibodies: Principles and Practice, Academic Press, pp. 59-103 (1986)). The hybridoma cells thus prepared are seeded and grown in a suitable culture medium that preferably contains one or more substances which inhibit the growth or survival of the unfused, parental myeloma cells. For example, if the parental myeloma cells lack the enzyme hypoxanthine guanine phosphoribosyl transferase (HGPRT), the culture medium for the hybridoma cells will typically include hypoxanthine, aminopterin, and thymidine (HAT medium), which prevent the growth of HGPRT-deficient cells.

Preferred myeloma cells are those that fuse efficiently, support stable high-level production of antibody by the selected antibody-producing cells, and/or are sensitive to a medium such as HAT medium. Examples of such preferred myeloma cell lines for the production of human monoclonal antibodies include, but are not limited to, murine myeloma lines such as those derived from MOPC-21 and MPC-11 mouse tumors (available from the Salk Institute Cell Distribution Center; San Diego, Calif.), SP-2 or X63-Ag8-653 cells (available from the American Type Culture Collection; Rockville, Md.), and human myeloma or mouse-human heteromyeloma cell lines (see, e.g., Kozbor, J. Immunol., 133:3001 (1984); and Brodeur et al., Monoclonal Antibody Production Techniques and Applications, Marcel Dekker, Inc., New York, pp. 51-63 (1987)).

The culture medium in which hybridoma cells are growing can be assayed for the production of monoclonal antibodies directed against the polypeptide of interest. Preferably, the binding specificity of monoclonal antibodies produced by hybridoma cells is determined by immunoprecipitation or by an in vitro binding assay, such as a radioimmunoassay (RIA) or an enzyme-linked immunoabsorbent assay (ELISA). The binding affinity of monoclonal antibodies can be determined using, e.g., the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, Academic Press, pp. 59-103 (1986)). Suitable culture media for this purpose include, for example, D-MEM or RPMI-1640 medium. In addition, the hybridoma cells may be grown in vivo as ascites tumors in an animal. The monoclonal antibodies secreted by the subclones can be separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody, to induce the synthesis of monoclonal antibodies in the recombinant host cells. See, e.g., Skerra et al., Curr. Opin. Immunol., 5:256-262 (1993); and Pluckthun, Immunol Rev., 130:151-188 (1992). The DNA can also be modified, for example, by substituting the coding sequence for human heavy chain and light chain constant domains in place of the homologous murine sequences (see, e.g., U.S. Pat. No. 4,816,567; and Morrison et al., Proc. Natl. Acad. Sci. USA, 81:6851 (1984)), or by covalently joining to the immunoglobulin coding sequence all or part of the coding sequence for a non-immunoglobulin polypeptide.

In a further embodiment, monoclonal antibodies or antibody fragments can be isolated from antibody phage libraries generated using the techniques described in, for example, McCafferty et al., Nature, 348:552-554 (1990); Clackson et al., Nature, 352:624-628 (1991); and Marks et al., J. Mol. Biol., 222:581-597 (1991). The production of high affinity (nM range) human monoclonal antibodies by chain shuffling is described in Marks et al., BioTechnology, 10:779-783 (1992). The use of combinatorial infection and in vivo recombination as a strategy for constructing very large phage libraries is described in Waterhouse et al., Nuc. Acids Res., 21:2265-2266 (1993). Thus, these techniques are viable alternatives to traditional monoclonal antibody hybridoma methods for the generation of monoclonal antibodies.Human Antibodies

As an alternative to humanization, human antibodies can be generated. In some embodiments, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

Alternatively, phage display technology (see, e.g., McCafferty et al., Nature, 348:552-553 (1990)) can be used to produce human antibodies and antibody fragments in vitro, using immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats as described in, e.g., Johnson et al., Curr. Opin. Struct. Biol., 3:564-571 (1993). Several sources of V-gene segments can be used for phage display. See, e.g., Clackson et al., Nature, 352:624-628 (1991). A repertoire of V genes from unimmunized human donors can be constructed and antibodies to a diverse array of antigens (including self-antigens) can be isolated essentially following the techniques described in Marks et al., J. Mol. Biol., 222:581-597 (1991); Griffith et al., EMBO 1, 12:725-734 (1993); and U.S. Pat. Nos. 5,565,332 and 5,573,905.

In certain instances, human antibodies can be generated by in vitro activated B cells as described in, e.g., U.S. Pat. Nos. 5,567,610 and 5,229,275.

6. Antibody Fragments

Various techniques have been developed for the production of antibody fragments.

Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, the antibody fragments can be isolated from the antibody phage libraries discussed above. Alternatively, Fab'-SH fragments can be directly recovered from E. coli cells and chemically coupled to form F(ab′)₂ fragments (see, e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870. Such linear antibody fragments may be monospecific or bispecific.

7. Antibody Purification

When using recombinant techniques, antibodies can be produced inside an isolated host cell, in the periplasmic space of a host cell, or directly secreted from a host cell into the medium. If the antibody is produced intracellularly, the particulate debris is first removed, for example, by centrifugation or ultrafiltration. Carter et al., BioTech., 10:163-167 (1992) describes a procedure for isolating antibodies which are secreted into the periplasmic space of E. coli. Briefly, cell paste is thawed in the presence of sodium acetate (pH 3.5), EDTA, and phenylmethylsulfonylfluoride (PMSF) for about 30 min. Cell debris can be removed by centrifugation. Where the antibody is secreted into the medium, supernatants from such expression systems are generally concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from cells can be purified using, for example, hydroxylapatite chromatography, gel electrophoresis, dialysis, and affinity chromatography. The suitability of protein A as an affinity ligand depends on the species and isotype of any immunoglobulin Fc domain that is present in the antibody. Protein A can be used to purify antibodies that are based on human γ1, γ2, or γ4 heavy chains (see, e.g., Lindmark et al., J. Immunol. Meth., 62:1-13 (1983)). Protein G is recommended for all mouse isotypes and for human γ3 (see, e.g., Guss et al., EMBO 1, 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker; Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

8. Bispecific Antibodies

Bispecific antibodies are antibodies that have binding specificities for at least two different epitopes. Bispecific antibodies can be prepared as full-length antibodies or antibody fragments (e.g., F(ab′)₂ bispecific antibodies).

Methods for making bispecific antibodies are known in the art. Traditional production of full-length bispecific antibodies is based on the co-expression of two immunoglobulin heavy chain-light chain pairs, where the two chains have different specificities (see, e.g., Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually performed by affinity chromatography. Similar procedures are disclosed in PCT Publication No. WO 93/08829 and Traunecker et al., EMBO 1, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding present in at least one of the fusions. DNA encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains into one expression vector when the expression of at least two polypeptide chains in equal ratios results in high yields or when the ratios are of no particular significance.

In some embodiments of this approach, the bispecific antibodies are composed of a hybrid immunoglobulin heavy chain with a first binding specificity (e.g., a first binding specificity for an epitope in C-reactive protein) in one arm, and a hybrid immunoglobulin heavy chain-light chain with a second binding specificity in the other arm. This asymmetric structure facilitates the separation of the desired bispecific compound from unwanted immunoglobulin chain combinations, as the presence of an immunoglobulin light chain in only one half of the bispecific molecule provides for a facile way of separation. See, e.g., PCT Publication No. WO 94/04690 and Suresh et al., Meth. Enzymol., 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side-chains from the interface of the first antibody molecule are replaced with larger side chains (e.g., tyrosine or tryptophan). Compensatory “cavities” of identical or similar size to the large side-chain(s) are created on the interface of the second antibody molecule by replacing large amino acid side-chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies can be made using any convenient cross-linking method. Suitable cross-linking agents and techniques are well-known in the art, and are disclosed in, e.g., U.S. Pat. No. 4,676,980.

Suitable techniques for generating bispecific antibodies from antibody fragments are also known in the art. For example, bispecific antibodies can be prepared using chemical linkage. In certain instances, bispecific antibodies can be generated by a procedure in which intact antibodies are proteolytically cleaved to generate F(ab′)2 fragments (see, e.g., Brennan et al., Science, 229:81 (1985)). These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody.

In some embodiments, Fab′-SH fragments can be directly recovered from E. coli and chemically coupled to form bispecific antibodies. For example, a fully humanized bispecific antibody F(ab′)₂ molecule can be produced by the methods described in Shalaby et al., J. Exp. Med., 175: 217-225 (1992). Each Fab' fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. See, e.g., Kostelny et al., J. Immunol., 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (VH) connected to a light chain variable domain (VL) by a linker which is too short to allow pairing between the two domains on the same chain. Accordingly, the VH and VL domains of one fragment are forced to pair with the complementary VL and VH domains of another fragment, thereby forming two antigen binding sites. Another strategy for making bispecific antibody fragments by the use of single-chain Fv (sFv) dimers is described in Gruber et al., J. Immunol., 152:5368 (1994).

Antibodies with more than two valencies are also contemplated. For example, trispecific antibodies can be prepared. See, e.g., Tutt et al., J. Immunol., 147:60 (1991).

In some embodiments, the normal concentration of C-reactive protein in the blood is below 3 mg/L. In some embodiments, an elevated concentration of C-reactive protein in the blood is at least 10 mg/mL or at least 15 mg/L. In certain embodiments, an elevated concentration of C-reactive protein in the blood is at least 30 mg/L.

9. C-reactive Protein

C-reactive Protein is a pentameric protein found in the blood plasma, whose circulating concentrations rise in response to inflammation. The protein is synthesized by the liver in response to factors released by macrophages and fat cells (adipocytes). The C-reactive protein gene is located on chromosome 1 (1q23.2). Each monomer of its pentameric structure has 224 amino acids, and a molecular mass of 25,106 Da. In serum, it assembles into stable pentameric structure with a discoid shape.

C-reactive protein is an acute-phase protein of hepatic origin that increases following interleukin-6 (IL-6) secretion by macrophages and T cells. Other inflammatory mediators that can increase C-reactive protein level are TGF-β1 and TNF-α. IL-6 is produced by macrophages, as well as adipocytes, in response to a wide range of acute and chronic inflammatory conditions, such as bacterial, viral, or fungal infections, rheumatic and other inflammatory diseases, malignancy; and tissue injury and necrosis. These conditions cause release of IL-6 and other cytokines that trigger the synthesis of C-reactive protein and fibrinogen by the liver. C-reactive protein binds to lysophosphatidylcholine expressed on the surface of dead or dying cells (and some types of bacteria) in order to activate the complement system via C1q and promote phagocytosis by macrophages, which clears necrotic and apoptotic cells and bacteria.

In healthy adults, the normal concentration of C-reactive protein is generally below 3.0 mg/L, e.g., between 0.8 mg/L to 3.0 mg/L. When there is a stimulus, the C-reactive protein level can increase dramatically, e.g., at least 5-fold (e.g., at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1,000-fold) more than its normal level. The plasma half-life of C-reactive protein is about 19 hours, and is constant in all medical conditions. Therefore, the only factor that affects the blood C-reactive protein concentration is its production rate, which increases with inflammation, infection, trauma, necrosis, malignancy, and allergic reactions.

In certain aspects, the methods described herein are used to measure and/or detect C-reactive protein. In certain aspects, the concentration or level of C-reactive protein is measured. In certain aspects, the biological sample in which C-reactive protein is measured is whole blood.

In certain aspects, the normal control concentration of C-reactive protein or reference value is below 3 mg/L (e.g., 2.8 mg/L, 2.6 mg/L, 2.4 mg/L, 2.2 mg/L, 2 mg/L, 1.8 mg/L, 1.6 mg/L, 1.4 mg/L, 1.2 mg/L, 1 mg/L, 0.8 mg/L, 0.6 mg/L, 0.4 mg/L, or 0.2 mg/L).

In certain aspects, the concentration of C-reactive protein in the biological sample is deemed elevated when it is at least 10% to about 60% greater than the normal control concentration of C-reactive protein. In certain aspects, the concentration of C-reactive protein in the biological sample is deemed elevated when it is at least about 10%, 15%, 20%, 25%, 30%, 35%, 40%, 45%, 50%, 55%, and/or 60% greater than the normal control concentration of C-reactive protein. In some embodiments, the concentration of C-reactive protein in the biological sample is deemed elevated when it is at least 5-fold (e.g., at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1,000-fold) more than its normal level.

In certain aspects, the concentration of C-reactive protein in the biological sample is deemed elevated when it is at least 10 mg/mL, at least 15 mg/L (e.g., at least 20 mg/L, 30 mg/L, 40 mg/L, 50 mg/L, 60 mg/L, 70 mg/L, 80 mg/L, 90 mg/L, 100 mg/L, 110 mg/L, 120 mg/L, 130 mg/L, 140 mg/L, 150 mg/L, 160 mg/L, 170 mg/L, 180 mg/L, 190 mg/L, or 200 mg/L). In certain aspects, the concentration of C-reactive protein in the biological sample is deemed elevated when it is at least 30 mg/L (e.g., at least 35 mg/L, 40 mg/L, 50 mg/L, 60 mg/L, 70 mg/L, 80 mg/L, 90 mg/L, 100 mg/L, 110 mg/L, 120 mg/L, 130 mg/L, 140 mg/L, 150 mg/L, 160 mg/L, 170 mg/L, 180 mg/L, 190 mg/L, or 200 mg/L).

In certain aspects, the methods herein include detecting the level of CRP in a subject experiencing discomfort. CRP is a protein produced primarily by the liver during an acute inflammatory process and other diseases. A positive test result indicates the presence, but not the cause, of the disease. The synthesis of CRP is initiated by antigen-immune complexes, bacteria, fungi, and trauma. CRP is functionally analogous to immunoglobulin G, except that it is not antigen specific.

10. Human Myxovirus Resistance Protein A (MxA)

“Human myxovirus resistance protein A” (MxA) the product of the MX1 gene, is a 76-kDa protein consisting of 662 amino acid residues and belonging to the dynamic superfamily of large GTPase. MxA Protein plays an important role in antiviral activity in cells against a wide variety of viruses, including influenza, parainfluenza, measles, coxsackie, hepatitis B virus, and Thogoto virus. The viruses are inhibited by MxA protein at an early stage in their life cycle, soon after host cell entry and before genome amplification.

In healthy adults, the normal concentration of MxA protein is about 2.0 ng/mL. Concentrations greater than about >40 ng/mL typically indicate a viral infection. In other aspects, concentrations greater than about 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, 250 ng/mL, 255 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, and/or 400 ng/mL indicate infection.

In some embodiments, the concentration of MxA protein in the biological sample is deemed elevated when it is at least 5-fold (e.g., at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1,000-fold) more than its normal level.

III. Methods

It is important to know whether bacteria or viruses cause an infection, because the treatments differ. Examples of bacterial infections include whooping cough, strep throat, ear infection and urinary tract infection (UTI). Viral infections include the common cold, flu, most coughs and bronchitis, chickenpox and HIV/AIDS.

The present disclosure can be used to distinguish between a bacterial infection and a viral infection. In most instances, an increase in CRP levels indicates a bacterial infection. Typically, a bacterial infection will not increase the levels of MxA. An increase in CRP levels indicates that one of more of the following is present in the subject: an acute, noninfectious inflammatory reaction (e.g., arthritis, acute rheumatic fever, Reiter syndrome, Crohn disease); a collagen-vascular diseases (e.g., vasculitis syndrome, lupus erythematosus); tissue infarction or damage (e.g., acute myocardial infarction {AMI}, pulmonary infarction, kidney or bone marrow transplant rejection, soft-tissue trauma); bacterial infections such as postoperative wound infection, urinary tract infection, or tuberculosis; malignant disease;

bacterial infection (e.g., tuberculosis, meningitis); and or increased risk for cardiovascular ischemic events.

A common type of bacterial pneumonia is called pneumococcal pneumonia. Pneumococcal pneumonia is caused by Streptococcus pneumoniae. Other bacterial types of pneumonia, include: mycoplasma pneumoniae, chlamydophila pneumoniae, and legionella pneumophila.

A viral infection will increase MxA levels. Diseases caused by viruses include chickenpox, HIV and the common cold. Other viral infections are caused by the following viruses: RSV, adenovirus, influenza A, herpes simplex, EBV, and parainfluenza. The influenza virus is the most common cause of viral pneumonia in adults. Respiratory syncytial virus (RSV) is the most common cause of viral pneumonia in children.

It is often challenging to differentiate viral from bacterial infections. This is especially true in young children that cannot verbalize their symptoms and in the outpatient setting where access to laboratory diagnostics is expensive, time consuming, and requires several days to produce a result. The methods herein can be especially useful with pediatric patients and subjects to differential or aid in the diagnosis of a bacterial infection versus a viral infection.

In certain aspects, the marker for viral infection is MxA and the marker for bacterial infection is C-reactive protein (CRP). High MxA protein levels are strongly correlated with systemic viral infection and increased CRP is more associated with bacterial infections. The present disclosure includes a rapid infectious screening test for identifying MxA and CRP in biological samples. MxA is present in leukocytes (white blood cells). Therefore, the sample can be taken anywhere leukocytes are available, for example in a peripheral blood sample, nasopharyngeal aspirates, tears, spinal fluid, and middle ear aspirates.

In certain aspects, measuring MxA and CRP together is better than measuring each of the two markers alone, i.e., the combination is more sensitive and/or specific at identifying both viral infection and bacterial infection. In certain aspects, low cut-off values of CRP show high sensitivity and low specificity for detecting bacterial infection. In certain aspects, high cut-off values of CRP show low sensitivity and high specificity for detecting bacterial infection. MxA is specific to identify viral infection, but it is not sensitive for bacterial infection. In certain aspects, multiplexing CRP and MxA including cut-off levels of low CRP, high CRP, and MxA together or in combination provide a sensitive and specific way to identify an immune response to a viral and/or bacterial infection.

Advantageously, the present disclosure provides technology that (i) accurately differentiates between a bacterial and viral infections; (ii) produces rapid results; (iii) is be able to differentiate between pathogenic and non-pathogenic bacteria that are part of the body's natural flora; (iv) differentiate between mixed co-infections and pure viral infections and (v) be applicable in cases where the pathogen is inaccessible (e.g. sinusitis, pneumonia, otitis-media, bronchitis, etc).

The disclosure provides a treatment recommendation (i.e., selecting a treatment regimen) for a subject by identifying the type infection (i.e., bacterial, viral, mixed infection or no infection) in the subject according to any of the disclosed methods and recommending that the subject receive an antibiotic treatment if the subject is identified as having bacterial infection or a mixed infection; or an anti-viral treatment is if the subject is identified as having a viral infection.

In another embodiment, the methods of the disclosure can be used to prompt additional targeted diagnosis such as pathogen specific PCRs, chest-X-ray, cultures etc. For example, a reference value that indicates a viral infection, may prompt the usage of additional viral specific multiplex-PCRs, whereas a reference value that indicates a bacterial infection may prompt the usage of a bacterial specific multiplex-PCR. Thus, one can reduce the costs of unwarranted expensive diagnostic tests.

IV. Device

Various instruments and devices are suitable for use in the present disclosure. Many spectrophotometers have the capability to measure fluorescence. Fluorescence is the molecular absorption of light energy at one wavelength and its nearly instantaneous re-emission at another, longer wavelength. Some molecules fluoresce naturally, and others must be modified to fluoresce.

A fluorescence spectrophotometer or fluorometer, fluorospectrometer, or fluorescence spectrometer measures the fluorescent light emitted from a sample at different wavelengths, after illumination with light source such as a xenon flash lamp. Fluorometers can have different channels for measuring differently-colored fluorescent signals (that differ in their wavelengths), such as green and blue, or ultraviolet and blue, channels. In one aspect, a suitable device includes an ability to perform a time-resolved fluorescence resonance energy transfer (FRET) experiment.

Suitable fluorometers can hold samples in different ways, including cuvettes, capillaries, Petri dishes, and microplates. The assays described herein can be performed on any of these types of instruments. In certain aspects, the device has an optional microplate reader, allowing emission scans in up to 384-well plates. Others models suitable for use hold the sample in place using surface tension.

Time-resolved fluorescence (TRF) measurement is similar to fluorescence intensity measurement. One difference, however, is the timing of the excitation/measurement process. When measuring fluorescence intensity, the excitation and emission processes are simultaneous: the light emitted by the sample is measured while excitation is taking place. Even though emission systems are very efficient at removing excitation light before it reaches the detector, the amount of excitation light compared to emission light is such that fluorescent intensity measurements exhibit elevated background signals. The present disclosure offers a solution to this issue. Time resolve FRET relies on the use of specific fluorescent molecules that have the property of emitting over long periods of time (measured in milliseconds) after excitation, when most standard fluorescent dyes (e.g., fluorescein) emit within a few nanoseconds of being excited. As a result, it is possible to excite cryptate lanthanides using a pulsed light source (e.g., Xenon flash lamp or pulsed laser), and measure after the excitation pulse.

As the donor and acceptor fluorescent compounds attached to the antibodies move closer together, an energy transfer is caused from the donor compound to the acceptor compound, resulting in a decrease in the fluorescence signal emitted by the donor compound and an increase in the signal emitted by the acceptor compound, and vice-versa. The majority of biological phenomena involving interactions between different partners will therefore be able to be studied by measuring the change in FRET between two fluorescent compounds coupled with compounds which will be at a greater or lesser distance, depending on the biological phenomenon in question.

The FRET signal can be measured in different ways: measurement of the fluorescence emitted by the donor alone, by the acceptor alone or by the donor and the acceptor, or measurement of the variation in the polarization of the light emitted in the medium by the acceptor as a result of FRET. One can also include measurement of FRET by observing the variation in the lifetime of the donor, which is facilitated by using a donor with a long fluorescence lifetime, such as rare earth complexes (especially on simple equipment like plate readers). Furthermore, the FRET signal can be measured at a precise instant or at regular intervals, making it possible to study its change over time and thereby to investigate the kinetics of the biological process studied.

In certain aspects, the device disclosed in PCT/IB2019/051213, filed Feb. 14, 2019 is used, which is hereby incorporated by reference. That disclosure in that application generally relates to analyzers that can be used in point-of-care settings to measure the absorbance and fluorescence of a sample with minimal or no user handling or interaction. The disclosed analyzers provide advantageous features of more rapid and reliable analyses of samples having properties that can be detected with each of these two approaches. For example, it can be beneficial to quantify both the fluorescence and absorbance of a blood sample being subjected to a diagnostic assay. In some analytical workflows, the hematocrit of a blood sample can be quantified with an absorbance assay, while the signal intensities measured in a FRET assay can provide information regarding other components of the blood sample.

One apparatus disclosed in PCT/IB2019/051213 is useful for detecting an emission light from a sample, and absorbance of a transillumination light by the sample, which comprises a first light source configured to emit an excitation light having an excitation wavelength. The apparatus further comprises a second light source configured to transilluminate the sample with the transillumination light. The apparatus further comprises a first light detector configured to detect the excitation light, and a second light detector configured to detect the emission light and the transillumination light. The apparatus further comprises a dichroic mirror configured to (1) epi-illuminate the sample by reflecting at least a portion of the excitation light, (2) transmit at least a portion of the excitation light to the first light detector, (3) transmit at least a portion of the emission light to the second light detector, and (4) transmit at least a portion of the transillumination light to the second light detector.

One suitable cuvette for use in the present disclosure is disclosed in PCT/M2019/051215, filed Feb. 14, 2019. One of the provided cuvettes comprises a hollow body enclosing an inner chamber having an open chamber top. The cuvette further comprises a lower lid having an inner wall, an outer wall, an open lid top, and an open lid bottom. At least a portion of the lower lid is configured to fit inside the inner chamber proximate to the open chamber top. The lower lid comprises one or more (e.g., two or more) containers connected to the inner wall, wherein each of the containers has an open container top. In certain aspects, the lower lid comprises two or more such containers. The lower lid further comprises a securing means connected to the hollow body. The cuvette further comprises an upper lid wherein at least a portion of the upper lid is configured to fit inside the lower lid proximate to the open lid top.

### V. EXAMPLES

### Example 1

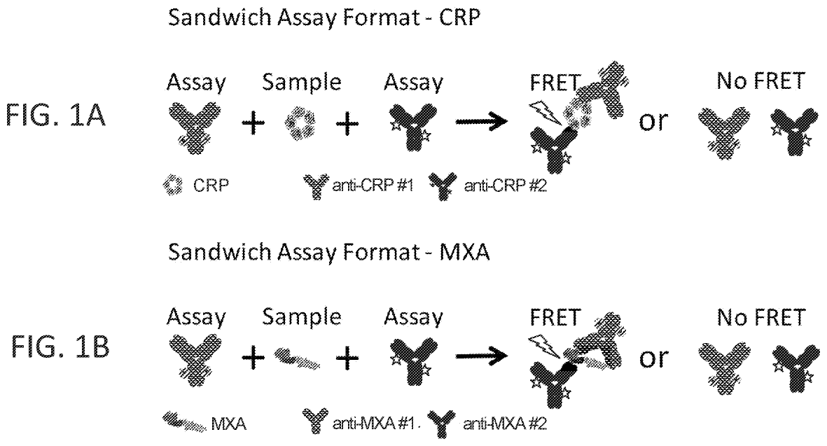

This example illustrates a method of this disclosure detecting the presence and amounts of C-reactive protein and MxA protein in a TR-FRET assay. FIGS. 1A-1B illustrate a sandwich assay method for detecting the presence or amount of C-Reactive Protein (CRP, FIG. 1A) and Myxovirus resistance protein 1 (MxA, FIG. 1B) in a sample. The assay includes contacting the sample with a first anti-CRP antibody having a first binding epitope to CRP, wherein the first anti-CRP antibody is labeled with a first donor fluorophore. Next, the assay includes contacting the sample with a second anti-CRP antibody having a second binding epitope to CRP, wherein the second anti-CRP antibody is labeled with a first acceptor fluorophore. In addition, the assay includes contacting the sample with a first anti-MxA antibody having a first binding epitope to MxA, wherein the anti-MxA antibody is labeled with a second donor fluorophore. Next, the assay includes contacting the sample with a second anti-MxA antibody having a second binding epitope to MxA, wherein the second anti-MxA antibody is labeled with a second acceptor fluorophore.

The sample is incubated for a time sufficient to obtain dual labeled CRP and dual labeled MxA; and then exciting the sample having dual labeled CRP and dual labeled MxA using one or more light sources to detect at least one fluorescence emission signal associated with fluorescence resonance energy transfer (FRET), wherein the first and second acceptor fluorophores are different.

### Example 2

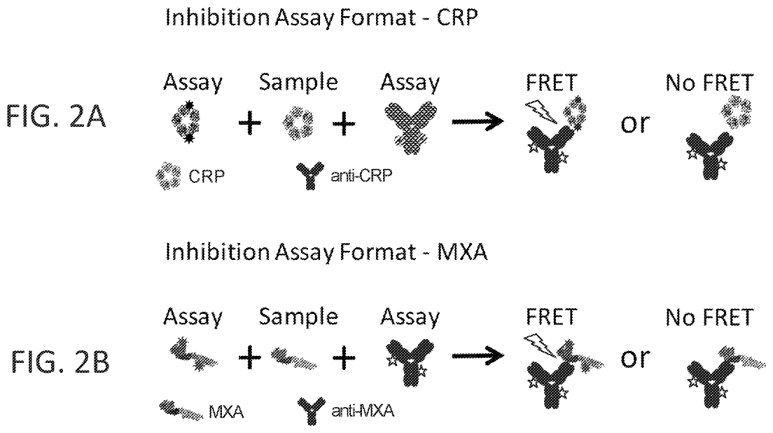

This example illustrates a method of this disclosure detecting the presence and amounts of C-reactive protein and MxA protein in a TR-FRET assay. As shown in FIG. 2A, an isolated C-reactive protein (CRP) labeled with a donor fluorophore binds to an anti-C-reactive protein antibody (MAB-1) labeled with an acceptor fluorophore. The C-reactive protein analyte is in a sample from a patient (i.e., whole blood sample) and it binds to anti-C-reactive protein antibody labeled with the acceptor fluorophore, thus, disrupting the FRET signal. In the presence of a high amount of C-reactive protein, the FRET signal is low, since the C-reactive protein in the sample (e.g., a whole blood sample) blocks or competes with the binding of the isolated C-reactive protein to the anti-C-reactive protein antibody.

Similarly, as shown in FIG. 2B, an isolated MxA protein (MxA) labeled with a donor fluorophore binds to an anti-MxA antibody (MAB-1) labeled with an acceptor fluorophore. The MxA protein analyte is in a sample from a patient (i.e., whole blood sample) and it binds to anti-MxA antibody labeled with the acceptor fluorophore, thus, disrupting the FRET signal. In the presence of a high amount of MxA, the FRET signal is low, since the MxA protein in the sample (e.g., a whole blood sample) blocks or competes with the binding of the isolated MxA protein to the anti-MxA antibody.

The decrease in each FRET signal is proportional to the level of C-reactive protein present and the level of MxA protein present in the patient's blood. FIG. 3A shows known amount of C-reactive protein as a control.

Donor fluorophore, Lumi4-Tb (also called Tb-H22TRENIAM-5LIO-NHS, FIG. 4), can be used to label an isolated C-reactive protein (CRP). Lumi4 has 4 spectrally distinct peaks, at about 490 nm, about 545 nm, about 580 nm, and about 620 nm, which can be used for energy transfer (FIG. 5). The acceptor fluorophores that can be used include but are not limited to: AlexaFluor 488, AlexaFluor 546, AlexaFluor 647 (FIG. 5), allophycocyanin (APC), and phycoerythrin (PE). Donor and acceptor fluorophores can be conjugated to antibodies using primary amines on antibodies.

The sequence of C-reactive protein (UniProt ID NO. P02741) is recited in SEQ ID NO:1.

Human C-reactive protein is available from lifediagnostics Catalog# 8000. C-Reactive Protein is available from R&D Systems catalog #1707-CR-200.

Human MxA is 662 amino acids (aa) in length having UniProt ID NO: P20591-1, and set forth as SEQ ID NO:2.

Human MxA is 662 amino acids available from NKMAX catalog #ATGP2826; Abnova catalog # LS-G3041 and OriGene, catalog: TP307418.

### Example 3

Using a ratio of at least 2 of the following: CRP, MX1 or PCT, to discern an acute viral infection from a bacterial infection using FRET within a homogeneous solution.

CRP is an acute inflammation protein whose concentration is dependent on the type and severity of the acute inflammation. It has been widely documented that CRP levels tend to be lower in viral infections compared to bacterial infections. Although a general difference in CRP concentration is observed between viral infections and bacterial infections, a universal cutoff that is both specific and sensitive has proven elusive using CRP levels alone.

To assist in differentiating bacterial infections from viral infections, an additional protein, MX1 (MXA) can be used. The MX1 protein is upregulated during viral infections and can be used in conjunction with CRP levels to discern, diagnose or differentiate an acute viral infection from a bacterial infection.

The current embodiment uses both the CRP and MX1 concentrations within human whole blood, plasma or serum to differentiate a viral from bacterial infection for a given patient or subject. The ratio from at least these two markers can be used to aid in discerning a bacterial from viral infection.

The claimed methodology of measuring MX1 utilizes a FRET method of detection within a homogeneous solution. There are two different MX1 assay formats claimed. One is an inhibition assay where the MX1 protein and an anti-MX1 antibody are used. For this format, the MX1 protein is either labeled with a donor or acceptor molecule and the anti-MX1 antibody is either labeled with a donor or acceptor molecule. If no MX1 protein is present within a sample, the MX1 labeled protein which binds to the anti-MX1 antibody bringing the donor and acceptor molecules close together creating a FRET signal. As the level of MX1 increases within a sample, it inhibits the labeled MX1 and anti-MX1 antibodies from binding reducing the observed signal from the FRET reaction. A depiction of the MX1 inhibition assay is shown in FIG. 2B.

In the competitive format, the conditions are as follows: the donor: Mx1-L4; the acceptor: Anti-Mx1-AF488; the assay buffer: TBS, 10% Glycerol, 0.1% BSA, 0.05% Tween.

A standard curve from the MX1 inhibition (competitive) format is shown in FIG. 3C, using the following tabulated data.

| MxA μ/mL | R/R0 |
|---|---|
| 25.000 | 0.32 |
| 6.250 | 0.43 |
| 1.563 | 0.72 |
| 0.391 | 0.89 |
| 0.098 | 0.97 |
| 0.024 | 0.99 |
| 0.006 | 0.94 |
| 0.000 | 1.00 |

Similarly, a second methodology of measuring MX1 is claimed that also utilizes a FRET method of detection within a homogeneous solution in a sandwich assay format. This format utilizes two anti-MX1 antibodies each bound to either a donor or acceptor molecule. If no MX1 is present within a sample the two labeled anti-MX1 antibodies will stay sufficiently far apart to not yield a FRET signal. As MX1 is introduced within a sample, the two anti-MX1 antibodies will bind to the MX1 protein allowing for a FRET signal to be observed. As the concentration of MX1 increases, so too does the FRET signal. A depiction of the claimed MX1 assay format is shown in FIG. 1B.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

SEQ ID NO: 1:
MEKLLCFLVLTSLSHAFGQTDMSRKAFVFPKESDTSYVSLKAPLTKPLKA
FTVCLHFYTELSSTRGYSIFSYATKRQDNEILIFWSKDIGYSFTVGGSEI
LFEVPEVTVAPVHICTSWESASGIVEFWVDGKPRVRKSLKKGYTVGAEAS
IILGQEQDSFGGNFEGSQSLVGDIGNVNMWDFVLSPDEINTIYLGGPFSP
NVLNWRALKYEVQGEVFTKPQLWP
SEQ ID NO: 2
MVVSEVDIAK ADPAAASHPL LLNGDATVAQ KNPGSVAENN
LCSQYEEKVR PCIDLIDSLR ALGVEQDLAL PAIAVIGDQS
SGKSSVLEAL SGVALPRGSG IVTRCPLVLK LKKLVNEDKW
RGKVSYQDYE IEISDASEVE KEINKAQNAI AGEGMGISHE
LITLEISSRD VPDLTLIDLP GITRVAVGNQ PADIGYKIKT
LIKKYIQRQE TISLVVVPSN VDIATTEALS MAQEVDPEGD
RTIGILTKPD LVDKGTEDKV VDVVRNLVFH LKKGYMIVKC
RGQQEIQDQL SLSEALQREK IFFENHPYFR DLLEEGKATV
PCLAEKLTSE LITHICKSLP LLENQIKETH QRITEELQKY
GVDIPEDENE KMFFLIDKVN AFNQDITALM QGEETVGEED
IRLFTRLRHE FHKWSTIIEN NFQEGHKILS RKIQKFENQY
RGRELPGFVN YRTFETIVKQ QIKALEEPAV DMLHTVTDMV
RLAFTDVSIK NFEEFFNLHR TAKSKIEDIR AEQEREGEKL
IRLHFQMEQI VYCQDQVYRG ALQKVREKEL EEEKKKKSWD
FGAFQSSSAT DSSMEEIFQH LMAYHQEASK RISSHIPLII
QFFMLQTYGQ QLQKAMLQLL QDKDTYSWLL KERSDTSDKR
KFLKERLARL TQARRRLAQF PG

What is claimed is:

1. A sandwich assay method for detecting the presence or amount of C-Reactive Protein (CRP) and Myxovirus resistance protein 1 (MxA) in a sample, the method comprising:
contacting the sample with a first anti-CRP antibody having a first binding epitope to CRP, wherein the first anti-CRP antibody is labeled with a first donor fluorophore;
contacting the sample with a second anti-CRP antibody having a second binding epitope to CRP, wherein the second anti-CRP antibody is labeled with a first acceptor fluorophore;
contacting the sample with a first anti-MxA antibody having a first binding epitope to MxA, wherein the anti-MxA antibody is labeled with a second donor fluorophore;
contacting the sample with a second anti-MxA antibody having a second binding epitope to MxA, wherein the second anti-MxA antibody is labeled with a second acceptor fluorophore;
incubating the sample for a time sufficient to obtain dual labeled CRP and dual labeled MxA; and
exciting the sample having dual labeled CRP and dual labeled MxA using one or more light sources to detect at least one fluorescence emission signal associated with fluorescence resonance energy transfer (FRET), wherein the first and second acceptor fluorophores are different.

2. The method of claim 1, wherein the first and second donor fluorophores are the same and the sample is excited using one light source.

3. The method of claim 1, wherein the first and second donor fluorophores are different and the sample is excited using two different light sources.

4. The method according to claim 1, wherein the FRET emission signals are time resolved FRET emission signals.

5. The method according to claim 1, wherein the sample is a biological sample.

6. The method according to claim 5, wherein the biological sample is selected from the group consisting of whole blood, urine, a fecal specimen, plasma, and serum.

7. The method according to claim 6, wherein the biological sample is whole blood.

8. The method according to claim 1, wherein the first donor fluorophore is a terbium cryptate.

9. The method according to claim 1, wherein the second donor fluorophore is a terbium cryptate.

10. The method according to claim 1, wherein the first acceptor fluorophore is selected from the group consisting of fluorescein-like (green zone), Cy5, DY-647, Alexa Fluor 488, Alexa Fluor 546, Alexa Fluor 647, allophycocyanin (APC), and phycoerythrin (PE).

11. The method according to claim 1, wherein the second acceptor fluorophore is selected from the group consisting of fluorescein-like (green zone), Cy5, DY-647, Alexa Fluor 488, Alexa Fluor 546, Alexa Fluor 647, allophycocyanin (APC), and phycoerythrin (PE).

12. The method according to claim 1, wherein the first acceptor fluorophore is Alexa Fluor 488 and the second acceptor fluorophore is Alexa Fluor 546.

13. The method according to claim 1, wherein the first acceptor fluorophore is Alexa Fluor 488 and the second acceptor fluorophore is Alexa Fluor 647.

14. The method according to claim 1, wherein the first acceptor fluorophore is Alexa Fluor 546 and the second acceptor fluorophore is Alexa Fluor 647.

15. The method according to claim 1, further comprising detecting the presence or amount of an additional biomarker.

16. The method according to claim 15, comprising:
contacting the sample with an additional antibody having a first binding epitope to the additional biomarker, wherein the additional antibody is labeled with a third donor fluorophore;
contacting the sample with a further antibody having a second binding epitope to the additional biomarker, wherein the further antibody is labeled with a third acceptor fluorophore;
incubating the sample for a time sufficient to obtain dual labeled additional biomarker; and
exciting the sample having dual labeled additional biomarker using a light source to detect two fluorescence emission signals associated with fluorescence resonance energy transfer (FRET), wherein the first, second, and third acceptor fluorophores are different.

17. The method according to claim 1, wherein the light source provides an excitation wavelength between about 300 nm to about 400 nm.

18. The method according to claim 1, wherein the fluorescence emission signals emit emission wavelengths that are between about 450 nm to 700 nm.

19. The method according to claim 1, wherein an elevated concentration of CRP in the blood is greater than or equal to 20 mg/mL.

20. The method according to claim 1, wherein an elevated concentration of MxA in the blood is greater than or equal to about 40 ng/mL.

21. An inhibition assay method for detecting the presence or amount of C-Reactive Protein (CRP) and Myxovirus resistance protein 1 (MxA) in a sample, the method comprising:
contacting the sample with a CRP complex comprising an anti-C-reactive protein antibody labeled with a first donor fluorophore and an isolated C-reactive protein labeled with a first acceptor fluorophore, wherein the CRP complex emits a fluorescence emission signal associated with fluorescence resonance energy transfer (FRET) when the first donor fluorophore is excited using a light source;
contacting the sample with a MxA complex comprising an anti-MxA antibody labeled with a second donor fluorophore and an isolated MxA protein labeled with a second acceptor fluorophore, wherein the MxA complex emits a fluorescence emission signal associated with fluorescence resonance energy transfer (FRET) when the donor fluorophore is excited using a light source;
incubating the sample with the CRP complex for a time sufficient for C-reactive protein in the sample to compete for binding to the anti-C-reactive protein antibody labeled with the first donor fluorophore;
incubating the sample with the MxA complex for a time sufficient for MxA protein in the sample to compete for binding to the anti-MxA antibody labeled with the second donor fluorophore; and
exciting the sample using a light source to detect a fluorescence emission signal associated with FRET, wherein an absence of the fluorescence emission signal or a decrease in the fluorescence emission signal relative to the fluorescence emission signal initially emitted by each of the complexes indicates the presence or amount of C-reactive protein and MxA protein in the sample.

22. A mixed sandwich-inhibition assay method for detecting the presence or amount of C-Reactive Protein (CRP) and Myxovirus resistance protein 1 (MxA) in a sample, the method comprising:
contacting the sample with a first anti-CRP antibody having a first binding epitope to CRP, wherein the first anti-CRP antibody is labeled with a first donor fluorophore;
contacting the sample with a second anti-CRP antibody having a second binding epitope to CRP, wherein the second anti-CRP antibody is labeled with a first acceptor fluorophore;
contacting the sample with a MxA complex comprising an anti-MxA antibody labeled with a second donor fluorophore and an isolated MxA protein labeled with a second acceptor fluorophore, wherein the MxA complex emits a fluorescence emission signal associated with fluorescence resonance energy transfer (FRET) when the second donor fluorophore is excited using a light source;
incubating the sample for a time sufficient to obtain dual labeled CRP;
incubating the sample with the MxA complex for a time sufficient for MxA protein in the sample to compete for binding to the anti-MxA antibody labeled with the second donor fluorophore; and
exciting the sample using a light source to detect a fluorescence emission signal associated with dual labeled CRP and wherein an absence of the fluorescence emission signal or a decrease in the fluorescence emission signal relative to the fluorescence emission signal initially emitted by the MxA complex indicates the presence or amount of MxA protein in the sample.

23. A mixed inhibition-sandwich assay method for detecting the presence or amount of C-Reactive Protein (CRP) and Myxovirus resistance protein 1 (MxA) in a sample, the method comprising:
contacting the sample with a CRP complex comprising an anti-C-reactive protein antibody labeled with a first donor fluorophore and an isolated C-reactive protein labeled with a first acceptor fluorophore, wherein the CRP complex emits a fluorescence emission signal associated with fluorescence resonance energy transfer (FRET) when the first donor fluorophore is excited using a light source;
contacting the sample with a first anti-MxA antibody having a first binding epitope to MxA, wherein the anti-MxA antibody is labeled with a second donor fluorophore;
contacting the sample with a second anti-MxA antibody having a second binding epitope to MxA, wherein the second anti-MxA antibody is labeled with a second acceptor fluorophore;
incubating the sample with the CRP complex for a time sufficient for C-reactive protein in the sample to compete for binding to the anti-C-reactive protein antibody labeled with the donor fluorophore;
incubating the sample for a time sufficient to obtain dual labeled MxA; and
exciting the sample using a light source to detect a fluorescence emission signal associated with dual labeled MxA and wherein an absence of the fluorescence emission signal or a decrease in the fluorescence emission signal relative to the fluorescence emission signal initially emitted by the CRP complex indicates the presence or amount of CRP in the sample.


SEQUENCE LISTING (2 sequences)

SEQ ID NO: 1 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 2 - protein, 662 aa, Homo sapiens
MVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKVNAFNQDITALMQGEETVGEEDIRLFTRLRHEFHKWSTIIENNFQEGHKILSRKIQKFENQYRGRELPGFVNYRTFETIVKQQIKALEEPAVDMLHTVTDMVRLAFTDVSIKNFEEFFNLHRTAKSKIEDIRAEQEREGEKLIRLHFQMEQIVYCQDQVYRGALQKVREKELEEEKKKKSWDFGAFQSSSATDSSMEEIFQHLMAYHQEASKRISSHIPLIIQFFMLQTYGQQLQKAMLQLLQDKDTYSWLLKERSDTSDKRKFLKERLARLTQARRRLAQFPG